(19) United States
(12) Patent Application Publication
(10) Pub. No.: US 2007/0004766 A1
(43) Pub. Date: Jan. 4, 2007

(54) Methods for relaxation of smooth muscle contractions using Trospium

(76) Inventor: A.K. Gunnar Aberg, Sarasota, FL (US)

Correspondence Address: NIELDS & LEMACK, 176 EAST MAIN STREET, SUITE 7, WESTBORO, MA, 01581, US

(21) Appl. No.: 11/474,154

(22) Filed: Jun. 23, 2006

Related U.S. Application Data
(60) Provisional application No. 60/696,271, filed on Jul. 1, 2005.

(51) Int. Cl. A61K 31/4747

(52) U.S. Cl. 514/278

(57) ABSTRACT

Methods are disclosed using Trospium Chloride, an antimuscarinic smooth muscle relaxant, for the treatment of urinary incontnence, while avoiding the concomitant liability of adverse side effect associated with other antimuscarinic drugs.

This application claims priority of Provisional Application Ser. No. 60/696,271 filed Jul. 1, 2005, the disclosure of which is hereby incorporated by reference.

### FIELD OF THE INVENTION

This invention relates to a compound named TROSPIUM, the possible prodrugs, salts and solvates thereof and the active metabolites of trospium and possible prodrugs, salts and solvates of said active metabolites. Trospium chloride has the formula:

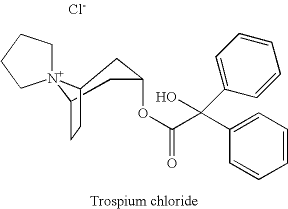

The USAN name Trospium Chloride (CAS-10405-02-4; INN) refers to an anticholinergic compound with the chemical name 3α-hydroxy-spiro[1αH,5αH-nortropane-8,1′-pyrrolidinium]chloride benzilate; C₂₅H₃₀ClNO₃; MW=427.97.

Trospium can be synthesized as described by Pfleger R. et al. in U.S. Pat. No. 3,480,626 and by Bertholdt H. et al. in Arzneimittel-Forschung, 1967, 17: 719-726. Lack of arrhythmogenic side effects of trospium was described by Aberg in U.S. Pat. No. 6,974,820.

Trospium chloride may be purchased from Galen Ltd, Craigavon, UK or from Madaus AG, Köln, Germany. Trospium can also be extracted from 20 mg trospium tablets (e.g. Regurin®, Madaus in Germany or Sanctura®, Indevus in the USA), using extraction methods commonly known to those skilled in the art. For use in pharmacological studies, Spasmolyt® (hoyer-madaus) tablets 20 mg were purchased. Thirty-nine tablets were ground in a mortar and the powder placed in a 150 ml beaker. Ethanol (50 ml) was added and the suspension was stirred at room temperature for 30 minutes. The suspension was filtered and the solid was stirred with ethanol and filtered as above five times. The combined ethanolic filtrates were concentrated to ca. 10 ml, and diethyl ether (ca. 20 ml) was added to give a cloudy suspension. After standing, the suspension was filtered to give 833 mg of colorless crystals. The crystals were dissolved in ethanol (ca. 20 ml) and diethyl ether (>20 ml) was added. After crystallization, the solid was filtered to give 412 mg of colorless product (white crystals), mp 220-225° C. NMR was consistent. HPLC showed 98.7% purity (Lot No. DA2-80).

Trospium has several known metabolites, the most well known being a spiroalcohol metabolite. The spiroalcohol metabolite has antimuscarinic activities that are believed to contribute to the therapeutic activity of trospium in vivo.

The present invention relates specifically to the therapeutic use of trospium, possible prodrugs thereof and the active metabolites of trospium and the possible prodrugs thereof, and pharmaceutical compositions containing at least one of said compounds for treating smooth muscle hyperactivity disorders, such as for example urinary disorders, including urinary incontinence and pollakiuria, and gastrointestinal disorders, including gastrointestinal hyperactivity, and other smooth muscle hyperactivity or hyperreactivity disorders including those occurring in conjunction with asthma, urolithiasis, cholelithiasis and choledocholithiasis, while avoiding the cardio-depressive effects of other anticholinergic compounds, such as for example oxybutynin.

The risk with cardio-depressive side effects of anticholinergic agents are particularly serious in patients with pre-existing cardiac disorders, in patients at risk for developing cardiac contractility disorders and in patients using other medication that may have cardio-depressive effects, such as for example anticholinergic drugs for urinary incontinence, (such as for example oxybutynin (Ditropan®, Alza), certain antihistamines (such as for example diphenhydramine (Benadryl®, Pfizer)), calcium antagonists (such as for example verapamil (Isoptin®, Abbott) or diltiazem (Cardizem®, Aventis) anti-arrhythmic drugs (such as for example mexiletine (Mexitil®, Boehringer-Ingelheim)) and adrenergic beta-receptor blocking drugs (such as for example propranolol (Inderal®, Wyeth). Individuals taking any type of medication that is causing, or that may be causing, cardio-depressive effects should not use a second drug with cardio-depressive side effects because of risk for additive cardiodepression and risk for acute heart failure.

### BACKGROUND OF THE INVENTION

Trospium has been shown to reduce bladder hyperactivity in patients suffering from urinary incontinence and exerts spasmolytic effects on the bladder by inhibiting the effects of acetylcholine on smooth muscle. Trospium has selectivity for muscarinic receptors over nicotinic receptors and as a result, no blocking effects are observed at skeletal neuromuscular junctions. Active metabolites of trospium exert antimuscarinic activities that are believed to account for part of the therapeutic activity of trospium in vivo.

In this document, the terms ‘anticholinergic’ and ‘antimuscarinic’ are interchangeable. The terms “inotropic”, “chronotropic” and “dromotropic” refer to force of cardiac contractions, heart rate and electric impulse conduction in the heart, respectively.

The term “cardiomyopathy” refers to a weakening of the heart muscle and often leads to changes in the heart muscle structure. Cardiomyopathies are often causing inadequate heart pumping ability and other heart function abnormalities.

The term “dyspnea” refers to shortness of breath, which symptom can be of pulmonary origin (pulmonary dyspnea) or of cardiac origin (cardiac dyspnea). Pulmonary congestion, caused by a cardiac contractile disorder, is often referred to as the mechanical component of cardiac dyspnea.

The terms “predisposed” to cardiac contractile disorder and “propensity” for developing cardiac contractile disorders, which terms are both used herein, refer to humans who are at risk for developing cardiac contractile disorders, such as for example elderly patients or patients suffering from certain diseases that may lead to cardiac contractile disorders, such as for example coronary atherosclerosis or myocardial infarct or patients who are smoking or who are obese or suffer from arterial hypertension or patients who have other cardiac risk factors or patients taking medication that can cause depression of cardiac contractility, such as for example patients taking adrenergic beta-receptors, such as for example propranolol (Inderal®, Wyeth), metoprolol (Lopressor®, Novartis).

The heart is a pump that supplies the peripheral organs with oxygen and nutrients. When the contractility of the heart is decreased, the heart does not adequately perform its function as a pump, which results in various abnormalities. Drugs that have negative effects on cardiac contractility will decrease the performance of the heart as a pump, which may be particularly serious in patients who are suffering from existing cardiac contractility disorders, such as for example heart failure or cardiomyopathy. Decreased cardiac contractility, which can be caused by various factors, such as for example medicines with cardio-depressive side effects, not only causes decreased pump functions of the heart, but decreased cardiac contractility can also lead to various cardiac diseases, such as for examples:

1. Aggravation of Left Ventricular Dysfunction, which most often is an abnormality of systolic (contraction phase of the heart beat) performance of the left ventricle of the heart.

2. Aggravation of Heart Failure, which is a clinical syndrome with abnormalities of myocardial contractility. The symptoms of heart failure may include fatigue, exercise limitation, dyspnea (shortness of breath) on exertion, orthopnea (shortness of breath relieved by sitting up), or paroxysmal nocturnal dyspnea (sudden shortness of breath at night).

3. Aggravation of Congestive Heart Failure, which is a clinical syndrome in which symptoms of Heart Failure are accompanied by symptoms of congestion (edema), such as for example peripheral edema (ankle swelling) or pulmonary congestion (rales in the lungs) and symptoms of dyspnea (shortness of breath).

Etiology of Cardiac Contractile Disorders

In individuals predisposed to cardiac contractile disorders, such as cardiomyopathy, heart failure or congestive heart failure, the disease usually begins with either decreased coronary blood flow (eventually causing angina pectoris or myocardial infarction), or ventricular overloads (e.g. systemic arterial hypertension) or loss and depression of cardiac tissue (e.g. from one or more cardiac infarcts, cardiomyopathies or from myocarditis).

Clinical Manifestation of Heart Failure

The most well known cardiac contractile disorders are heart failure and congestive heart failure. Major clinical manifestations of heart failure are left ventricular dysfunction, exercise intolerance, cardiac dyspnea and pulmonary congestion.

Chronic heart failure symptoms can be divided into four classes according to the New York Heart Association (NYHA) as follows:

Class 1: Symptoms cause no limitation of physical activity. Ordinary physical activity does not lead to undue fatigue, or dyspnea.

Class 2: Symptoms cause slight limitation of physical activities. Patient is comfortable at rest, but ordinary physical activity results in fatigue, palpitations, or dyspnea (shortness of breath).

Class 3: Symptoms cause marked limitation of physical activity. Patient is comfortable at rest, but even slight physical activity causes fatigue, palpitations, or dyspnea.

Class 4: Symptoms of cardiac insufficiency are present at rest, and discomfort is increased with any physical activity.

Numerous risk factors for the development of contractile disorders have been identified. Examples of risk factors in individuals who are predisposed for cardiac contractile diseases are:

Age:

Heart failure is the most common reason for hospitalization in the elderly, and as the population ages, the incidence of congestive heart failure is rising dramatically. Thus, the elderly are more disposed to cardiac contractile disorders, such as heart failures and cardiomyopathies, than are younger individuals. The term “elderly” is defined as being 65 years of age or older.

Gender:

Men are more disposed to developing cardiac contractile diseases, than women, although the difference narrows in elderly individuals.

Genetics:

A family history of congestive heart failure, particularly if caused by cardiomyopathies, is considered to predispose people to the disease.

High Blood Pressure (Hypertension):

High blood pressure is defined as a disease with arterial blood pressure readings of 140/80 mmHg or higher, with the exception that 130/80 mmHg is considered high blood pressure in people with diabetes or chronic kidney disease. Hypertension, can cause heart attacks but is also a major cause of cardiac contractile disorders even in the absence of a heart attack. About 75 percent of cases of heart failure start with hypertension. (Univ. of Maryland Med Center (www.umm.edu/heart/heart_failure/causes_risk). Thus individuals suffering from high blood pressure are considered predisposed to cardiac contractile disorders, such as for example heart failure.

Coronary Artery Disease:

Coronary artery disease is the end result of a complex process called atherosclerosis. It is the most common cause of heart attack and involves the buildup of unhealthy cholesterol on the arteries, with inflammation and injury in the cells of the blood vessels. The arteries narrow and become brittle and subject to damage. Heart failure in such cases most often results from a localized contractile defect in the left side of the heart. Thus, individuals suffering from coronary heart disease are predisposed to developing cardiac contractile disorders.

Cardiac Damage after a Heart Attack:

With modern treatment, most people survive heart attacks, but the physical damage done to the heart muscles by the attack often causes contractile disorders leading to heart failure. Thus, various types of cardiac damage by heart attacks are major factors in the dramatic increase in heart failure cases over the past decade and individuals who have had one or more heart attacks are considered to be predisposed for development of cardiac contractile disorders, such as for example heart failures or cardiac myopathies.

Pulmonary Congestion or Dyspnea:

Pulmonary congestion or dyspnea with risk for chronic pulmonary congestive disease is a well known risk factor for the development of congestive heart failure and possibly other cardiac contractile disorders, particularly if said pulmonary disorders are diagnosed in an elderly patient.

Chronic Alcohol Abuse:

Chronic alcohol abuse can damage the heart muscles, can cause hypertension, and may prove to be a cause of idiopathic dilated cardiomyopathy. Thus individuals who chronically abuse alcohol are also predisposed to heart failure, cardiomyopathies and other contractile cardiac diseases.

Smoking:

Smoking is a well-known risk factor, not only for the development of contractile disorders but also for other cardiovascular disorders, such as myocardial infarction and hypertension. Thus, long-term smokers are considered as being predisposed to various cardiac disorders, such as for example heart failure and other contractile disorders.

As known to those skilled in the art, numerous other factors can increase the risk for cardiac disorders, such as for example Obesity, Sedentary Living, Severe Emphysema, Hyperthyroidism, Thiamine (a vitamin B) Deficiency, Myocarditis (viral infection of the heart muscle).

The anticholinergic drugs that are being used for urinary incontinence (such as for example oxybutynin) have cardio-depressant activity and decrease cardiac contractility, which means that these drugs weaken the force of contractions of the heart (“negative inotropic activity”). This unwanted side effect of the presently used anticholinergic drugs is particularly serious since these drugs are used almost exclusively by elderly patients who suffer from urinary urge incontinence as their primary disease. Since these patients are mainly elderly, cardiac diseases, including heart failure and congestive heart failure or the risk thereof are prevalent. An estimated 17 million patients in the USA—mostly elderly patients—suffer from urinary urge incontinence and about 5 million Americans—mostly elderly patients—suffer from heart failure.

The term “smooth muscle disorder” in this document refers to a condition where smooth muscle (excluding vascular smooth muscle) contractility is increased in an individual, which means that said smooth muscle is expressing hyperactivity, including urinary incontinence such as urinary urge incontinence disorders, gastrointestinal smooth muscle hyperactivity disorders such as irritable bowel syndrome and diarrhea, kidney and gall bladder smooth muscle disorders such as urolithiasis, cholelithiasis and choledocholithiasis and pulmonary/bronchial smooth muscle hyperactivity disorders such as asthma, COPD and bronchitis.

The term “increased propensity for heart failure” in this document refers to increased risk for developing heart failure. The terms “decreased cardiac contractility”, “impaired cardiac contractility”, “negative inotropic activity”, “negative inotropic effect” and “cardiac contractility disorder” in this document refer to a condition where the cardiac contractility is decreased in an individual, which means that the force of cardiac contractions are less than normal for said individual, which in turn means that the heart is unable to sustain proper blood circulation in the body.

“Cardiac contractile diseases” can be of various types such as for example “heart failure”, “congestive heart failure”, “cardiac failure”, “acute heart failure”, “cardiac insufficiency”, “myocardial insufficiency” or “cardiomyopathies”. The term “Cardiac contractile disease” refer to mechanical inadequacies of the heart so that as a pump it fails to maintain the circulation of blood, with the result that symptoms such as congestion, dyspnea, edema and oxygen deprivation become evident.

“Congestive heart failure” is a chronic condition that is caused by decreased cardiac contractility and/or fluid accumulation. This condition is most often caused by a weakened heart that is not able to sustain a normal blood pressure and this condition is not seldom observed in patients following myocardial infarction and in patients suffering from advanced pulmonary congestion and dyspnea.

“Acute heart failure” refers to is an emergency situation that despite aggressive treatment often is fatal. Acute heart failure is most often seen in patients with chronically impaired myocardium and acutely decreased cardiac contractility. It is often the acute reason for death in patients suffering from massive heart infarcts.

“Heart failure” is a general term and refers to the inability of the heart to sustain arterial blood pressure at normal levels, thereby causing insufficient perfusion of peripheral organs. Many types of heart failure exist and many classifications of heart failure also exist. Textbooks in cardiology discuss the various types of heart failure in detail and reference is made to Braunwald, E (Editor): Heart Disease A Textbook in Cardiovascular Medicine, 4^{th} Edition, W.B. Saunders Company, Philadelphia; ISBN 0-7216-3096-0, which is hereby incorporated by reference.

Patients suffering from cardiac contractile disorders are often prescribed drugs that increase cardiac contractility, such as for example digitalis medicines, such as for example digoxin (Lanoxin®, Glaxo). The use of medications with cardio-depressant effects are contraindicated in patients suffering from heart failure, since the symptoms of heart failure may worsen because these drugs will further reduce the ability of the heart to pump blood. Drug-induced cardio-depression may cause acute heart failure in patients with compromised cardiac function, which is one reason why these patients should avoid medications with negative effects on cardiac contractility.

Patients who are predisposed for developing cardiac contractile disorders should avoid any drugs that may increase the risk for developing the disease, as known to those skilled in the art of medicine. Thus, myocardial remodeling (thickening of the ventricular walls, etc.) that is seen in patients with heart failure will develop faster if the heart is forced to work harder, such as is the case after loss of myocardial contractile tissues (e.g. from infarcts), increased arterial blood pressure (hypertension) or decreased cardiac contractility by cardiomyopathies or medications with negative effects on contractility.

Patients suffering from coronary atherosclerosis and impaired coronary blood flow may have impaired oxygenation of the heart and have therefore a propensity for developing angina pectoris, myocardial infarcts and heart failure and the use of cardio-depressant drugs can cause an increased burden on their hearts, which may already be compromised. Regardless if these patients suffer from diagnosed cardiomyopathies or heart failure, or if they are at risk for developing a cardiac contractile disorder, cardio-depressant medications should be avoided, as known to those skilled in the art of medicine.

It is a method of the present invention to determine if patients, who are suffering from smooth muscle disorders such as for example urinary urge incontinence, simultaneously suffer from or have a propensity for a cardiac contractile disorders and if said determination is positive, administering to said patients of a therapeutically effective amount of trospium or an active metabolite thereof or a pharmaceutically acceptable salt thereof. Said determinations of cardiac contractile disorders can be performed using measurements of heart rate and ECG, measurements of systolic and diastolic blood pressures, echocardiography, exercise stress testing, coronary arteriography, catheterization of the heart and of the pulmonary arteries and of the central veins and arteries, using methods that are known to those skilled in the art of medicine and physical examination. Those physicians who are skilled in the art of cardiovascular medicine may identify patients suffering from heart failure or patients at risk for developing heart failure from previous experience without performing extensive testing or measurements.

While anticholinergic medications for urinary urge incontinence, such as for example oxybutynin, have cardio-depressive effects, it has now unexpectedly been found that the anticholinergic drug trospium—contrary to other anticholinergic drugs for urinary incontinence—does not express any cardio-depressive side effects or significantly less cardiodepression than currently used drugs, such as oxybutynin and tolterodine.

No known reference teaches or enables the methods of the present invention comprising administering trospium to a human suffering from a cardio-depressive disorder or being at risk for developing cardio-depression; nor do the published references alone or in combination suggest these methods. It is of importance to note that particularly elderly patients may be at risk to develop cardio-depression, since the frequency of such disorders increase by age. It should also be kept in mind that urinary urge incontinence, which is the main clinical indication for trospium, is almost exclusively a disease of the elderly. The term “elderly” is in this document defined as an individual, who is 65 years of age or older. The terms “cardio-depressant drug” and “cardio-depressant activity” are in this document defined as causing negative effects on cardiac contractility (“negative inotropic effects”). Examples of drugs with cardio-depressant activity are adrenergic beta-receptor blockers, calcium antagonists, antiarrhythmic drugs with “local anesthetic effects” and any other drug—such as for example oxybutynin and tolterodine—that causes cardio-depression by “local anesthetic” mechanisms or by other mechanisms.

### SUMMARY OF THE INVENTION

Pharmacological studies of trospium have now been performed in comparison with known and marketed antimuscarinic drugs with therapeutic activity against cholinergically mediated diseases, such as for example forms of urinary incontinence.

The present studies have confirmed that trospium, as well as darifenacin, tolterodine and oxybutynin, have potent antimuscarinic activity.

It has now been found that while antimuscarinic drugs for incontinence, such as for example oxybutynin have cardio-depressive effects, trospium, surprisingly, does not cause this side effect at all or does not cause cardio-depressive effects when used in therapeutic doses.

It is well known by those skilled in the arts of pharmacology and toxicology that certain animal models, such as for example the measurement of cardiac contractility in isolated heart preparations is highly indicative of risk for cardio-depressive activity of drugs in vivo in humans. It has now surprisingly been found that trospium does not cause cardio-depressive effects in this, highly relevant laboratory animal model. Therefore, trospium will offer anticholinergic treatment for muscarinic disorders, including urinary voiding disorders such as urinary urge incontinence and including gastric motility disorders such as for example irritable bowel syndrome and diarrhea in humans, while avoiding the concomitant liability of cardio-depressant side effects that is associated with currently used antimuscarinic drugs. Trospium will therefore offer potential for anticholinergic treatment of smooth muscle disorders, including urinary incontinence such as urinary urge incontinence disorders, gastrointestinal smooth muscle disorders and kidney and gall bladder smooth muscle disorders, such as urolithiasis, cholelithiasis and choledocholithiasis, while being devoid of cardio-depressive side effects at therapeutic concentrations and while not causing worsening of cardio-depressive disorders of patients suffering from such disorders and being devoid of causing the expression of cardio-depressive disorders in patients at risk for expressing such disorders.

In cases where urgent anticholinergic treatment is preferred, trospium can be administered parenterally, such as by the intravenous route to rapidly alleviate the smooth muscle spasm and the intense smooth muscle pain in connection with urolithiasis or cholelithiasis or choledocholithiasis. In cases of severe pain, as for example in patients suffering from urolithiasis, trospium can be combined with drugs that are known to alleviate pain.

Since muscarinic receptors have been shown to play a significant role in the etiology of acute pancreatitis (Gronroos et al., Dig Dis 1992, 10: 38-45), trospium will be useful for the treatment of this disease, particularly in patients suffering from a cardiac contractility disorders or in patients predisposed to such cardiac disorders.

Urinary incontinence is a disease that in many patients is not consistent, but it is recurrent and patients often take their medication to prevent the re-occurrence of the symptoms of their disease. The magnitude of a prophylactic or a therapeutic dose of a compound of the present invention in the acute or chronic management of disease will vary with the severity and nature of the condition to be treated and with the route of administration. The dose and the frequency of the dosing will also vary according to the age, body weight, expected results and response of the individual patient. Doses as low as 1 mg to as high as 240 mg, as a single dose or divided into repeated doses, may be administered daily to patients in need of such therapy. More preferred are daily doses of 10 mg to 60 mg of trospium chloride, as a single daily dose or divided into repeated doses during a 24-hours period. Trospium can be administered as single daily doses or as repeated doses up to four times daily to patients in need of such therapy. In general, a therapeutically effective amount of the drug will be administered and an amount of the drug that is sufficient to treat the disorder but insufficient to cause adverse effects will be used. Most often, the daily oral dose of trospium is one 20-mg tablet twice daily to patients suffering from urinary urge incontinence. In managing the patient, the therapy may be initiated at a lower dose, perhaps at about 5 mg to about 10 mg, twice daily, and is usually increased up to 20 mg depending on the patient's global response. It may be necessary to use dosages outside these ranges, as will be apparent to those skilled in the art. Further, it is noted that the clinician or treating physician will know how and when to interrupt, adjust, or terminate therapy in conjunction with individual patient response.

The terms “a therapeutically effective amount” and “an amount sufficient to treat the disorder but insufficient to cause adverse effects” are encompassed by the above-described dosage amounts and dose frequency schedule.

Any suitable route of administration may be employed for providing the patient with an effective dose of the compounds of the present invention. For example, oral, sublingual, parenteral (i.e. subcutaneous, intramuscular, intravenous, etc.), transdermal, vaginal, aerosol and like forms of administration may be employed. Additionally, the drug may be administered directly into the bladder, as described for oxybutynin by Massad C. A., et al. in J. Urol., 1992, 148: 595-597 and for trospium by Schwantes U., et al. in U.S. Pat. No. 5,998,430 or rectally directly into the gastrointestinal canal. Dosage forms include tablets, troches, dispersions, suspensions, solutions, capsules, suppositories, microencapsulated formulations, slow-release and controlled release formulations, transdermal delivery systems, including, for example patches, creams, ointments and electrophoretic systems, mucosal or trans-mucosal formulations and the like. Controlled-release or slow-release oral formulations will be particularly useful, as realized by those skilled in the art.

Prodrugs of trospium or prodrugs of the active spiroalcohol metabolite can be prepared by those skilled in the art, as has been described for an active metabolite of tolterodine by Sparf B. et al. in EP 0957 073 A1, which is included hereby in its entirety.

The pharmaceutical compositions of the present invention comprise of trospium or an active metabolite thereof as the therapeutic ingredient, or any possible and pharmaceutically acceptable salts or solvates thereof, and said pharmaceutical formulations may also contain a pharmaceutically acceptable carrier, and optionally, additional therapeutic ingredients that offer benefits to the patient. Ester prodrugs of trospium or a metabolite of trospium may improve the oral availability of said compounds and can be made by those skilled in the art, for example, as described by Sparf et al. in EP 0957 073 A1.

The terms “pharmaceutically acceptable salts” or “pharmaceutically acceptable salt thereof” refer to possible salts of trospium and the active metabolite of trospium and said salts may be prepared from pharmaceutically acceptable acids. Suitable pharmaceutically acceptable salts for the compounds of the present invention include but are not limited to the chloride of trospium, but also other halogen salts and salts such as acetic, benzenesulfonic (besylate), benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, tartaric, and the like, if possible to synthesize and if pharmaceutically acceptable. Particularly preferred is Trospium Chloride.

The compositions of the present invention include suspensions, solutions, syrups, elixirs and solid dosage forms. Carriers such as starches, sugars, and microcrystalline cellulose, diluents, granulating agents, lubricants, binders, disintegrating agents, and the like are suitable in the case of oral solid preparations (such as powders, capsules, and tablets), and oral solid preparations are preferred over the oral liquid preparations. Because of their ease of administration, tablets and capsules represent the more advantageous oral dosage unit forms, in which case solid pharmaceutical carriers are employed. If desired, tablets may be coated, using standard aqueous or nonaqueous techniques. Capsules can be hard or soft.

In addition to the common dosage forms set out above, the compounds of the present invention may also be administered by controlled release means and delivery devices, such as for example patches or creams, to obtain improved pharmacokinetic profiles (such as sustained and stable plasma levels or prolonged duration of activity) and/or decreased side effects. The compounds of the present invention may also be administered by controlled-release tablet formulations of trospium or an active metabolite of trospium, allowing once-daily administration, which formulations have obvious advantages for the patient, as realized by those skilled in the art.

Pharmaceutical compositions of the present invention, suitable for oral administration, may be presented as discrete unit dosage forms such as capsules, cachets, suppositories, or tablets, or slow-release formulations, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the active ingredient with the carrier, which constitutes one or more necessary ingredients. In general, the compositions are prepared by uniformly admixing the active ingredient with liquid carriers or solid carriers or both, and then, if necessary, shaping the product into the desired presentation. For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made by molding, in a suitable machine, a mixture of the powdered compound moistened with an inert liquid diluent. All of the foregoing techniques are well know to persons of skill in the pharmaceutical art. Each tablet or capsule may contain from about 1 mg to about 60 mg of the active ingredient, an amount of 20 mg being preferred. An example of an oral unit dose formulation is shown below.

### EXAMPLE 1

Oral Unit Dosage Formulation (Tablets)

| | | per batch of |
|---|---|---|
| Ingredients | per tablet | 10,000 tablets |
| Trospium | 20 mg | 200 g |
| Microcrystalline cellulose | 30 mg | 300 g |
| Lactose | 70 mg | 700 g |
| Calcium separates | 2 mg | 20 g |
| FD&C Blue #1 Lake | 0.03 mg | 300 mg |

Trospium is blended with lactose and cellulose until a uniform blend is formed. The lake is added and further blended. Finally, the calcium stearate is blended in, and the resulting mixture is compressed into tablets using, for example, a 9/32 inch shallow concave punch. Tablets of other strengths may be prepared by altering the ration of active ingredient to the excipients or altering the final weight of the tablet.

Trospium may also be administered by transdermal delivery systems, such as for example cremes or transdermal patches. Penetration-enhancing compounds may have to be used.

### Pharmacological Studies of Trospium

1. Ligand Binding Studies: Affinity for Muscarinic Receptors.

Methods:

The experiments were carried out on membranes prepared from SF9 cells that expressed human recombinant muscarinic receptor subtypes. After incubation with the test article and the proper radioligand (³H scopolamine methylchloride) and washing, bound radioactivity was determined with a liquid scintillation counter, using a commercial scintillation cocktail. The specific radioligand binding to a muscarinic receptor was defined as the difference between total binding and nonspecific binding determined in the presence of an excess of an unlabelled ligand. IC₅₀ values (concentrations required to inhibit 50% of specific binding) were determined by non-linear regression analysis of the competition curves, from which affinity (pKi) values were determined (Cheng Y. et al. Biochem Pharmacol 1073, 22: 3099-3108.)

Results:

Affinity (negative logarithm of the dissociation constant Ki) of trospium and reference compounds for human recombinant receptors

| Test compound | M-1 | M-2 | M-3 | M-4 |
|---|---|---|---|---|
| Trospium | 9.1 | 9.2 | 9.3 | 9.0 |
| Tolterodine | 8.8 | 8.1 | 8.7 | 7.9 |
| Oxybutynin | 8.8 | 7.9 | 8.8 | 8.2 |
| Darifenacin | 8.3 | 7.7 | 9.2 | 7.4 |

Conclusions:

Trospium had slightly higher affinity for human muscarinic receptors than the reference compounds. The therapeutic activity of antimuscarinic drugs in overactive human bladders are generally considered to be related to affinity for M-2/M-3 receptors, while the side effect xerostomia (dry mouth) is due mainly, but not exclusively, to inhibition of M-1 receptors in salivary glands. In the present studies, darifenacin was the only compound demonstrating a statistically significant, albeit low, M-3/M-1 receptor selectivity.

2. Functional Characterization of Antimuscarinic and Antispasmodic Activities on Bladder Smooth Muscle Strips.

Methods:

Experiments have now been performed using methods described by Kachur et al, 1988, (J Pharmacol Exp Ther 247: 867-872) and Noronha-Blob et al. (J Pharmacol Exp Ther 256: 562-567). Strips of tissue (approximately 10 mm long and 1.5 mm wide) were removed from the urinary bladder of male guinea pigs. The tissues were suspended in an oxygenated buffer of the following composition, in mM: NaCl 133; KCl 4.7; CaCl₂ 2.5; MgSO₄ 0.6; NaH₂PO₄ 1.3; NaHCO₃ 16.3; and glucose 7.7. The smooth muscle strips were maintained at or about 37.5° C. in tissue chambers and allowed to equilibrate with the bathing solution for one hour before proceeding with the experiment. Contractions induced by carbachol were used to evaluate the anticholinergic activity of trospium and the reference compounds oxybutynin and tolterodine as described by Smith et al., 1998.

Results:

The antimuscarinic compounds terodiline, trospium, oxybutynin, des-oxybutynin, darifenacin and tolterodine potently inhibited carbachol-induced contractions with K_{B} values between 1.5 nM and 5.5 nM. There were no differences of biological significance between these test compounds, thereby confirming receptor binding studies in this functional test system.

3. Side Effects Concerning Cardiac Contractility.

Methods:

Male Sprague Dawley rats, weighing 250 to 280 g were used. The animals were euthanized and the heart was removed. Slices of atrial tissues were cut out of the isolated hearts and mounted in a bath containing a modified Kreb's solution of the following compositions, in mM: Na⁺, 137; K⁺, 6; Mg²⁺, 1.3; Ca²⁺, 2.2; Cl⁻, 134; HCO₃⁻, 15.4; H₂PO₄, 1.2 and glucose 11.2. The cardiac muscle strips were maintained at 37.0° C. in tissue chambers and allowed to equilibrate with the bathing solution before proceeding with the experiment. All tissues were pre-contracted before the tests to verify the viability of the tissues. To study the effects of test articles on cardiac contractility, predetermined concentrations (0.01, 0.05, 0.1 and 0.3 mmol/L) of trospium chloride or a reference compound (usually oxybutynin hydrochloride) were added to the baths and the cardiac strips were paced with an electric stimulator, kept at a setting of 120 beats per minute.

Results:

Results demonstrate that cardiac contractility was severely impaired by oxybutynin. There was no significant impairment of cardiac contractility by trospium. Typical results are shown in table I, where the contractility (force of cardiac muscle contractions) is expressed as percent of the pre-drug contractile force of each individual cardiac preparation.

TABLE I

| Conc. (mmol/L) | 0.01 | 0.05 | 0.1 | 0.3 |
|---|---|---|---|---|
| (Vehicle | Dose 1 | Dose 2 | Dose 3 | Dose 4) |
| Vehicle (%) | 98 ± 2 | 95 ± 5 | 90 ± 5 | 86 ± 7 |
| Trospium Chloride (%) | 96 ± 3 | 92 ± 5 | 81 ± 7 | 74 ± 8 |
| Oxybutynin HCl (%) | 83 ± 5 | 52 ± 7 | 27 ± 5 | 19 ± 5 |

Mean and SEM. N = 9.
Initial contractility in the absence of any drug = 100%.
There were no statistically significant difference effects of trospium, while oxybutynin significantly (P < 0.01) decreased cardiac contractility.

Conclusions:

The present results demonstrate that cardiac contractility was impaired by administration of oxybutynin, but not by trospium. It was found that trospium did not have unwanted side effects on cardiac contractility in concentrations as high as 0.3 mmol/L. It was concluded that oxybutynin, but not trospium is able to decrease cardiac contractility at therapeutic concentrations. As known by those skilled in the art of pharmacology, this method of evaluating cardiac contractility in heart tissues from rodents is a relevant model for studies of effects of drugs on cardiac contractility and is considered to be highly predictive of cardio-depressant drug effect in humans.

### Equivalents

Those skilled in the art will recognize, or be able to ascertain, using no more than routine experimentation, many equivalents to the specific embodiments of the invention described herein. Such equivalents include numerous possible pharmaceutically acceptable salt forms of trospium and the active metabolites thereof, or where appropriate one or other of the hydrate forms or solvates thereof. Such equivalents also include the co-administration of at least one compound of the present invention with any other compound or drug that may be used in combination with medication for urinary incontinence, intestinal smooth muscle hyperactivity, or bronchial smooth muscle hyperactivity. Those skilled in the art of medicine will also realize that higher or lower doses than those indicated here may be preferred and the doses may be given more or less frequently than suggested here.

The spiroalcohol metabolite of trospium has antimuscarinic activity and is therefore having therapeutic activity in patients suffering from conditions of smooth muscle hyperactivity. All active metabolites of trospium are included in this present invention.

Those skilled in the art, will realize that smooth muscle motility disorders, include urinary bladder disorders, as for example urinary incontinence diseases of various types (urge incontinence, stress incontinence, etc.), disorders of the gastrointestinal tract, as for example gastric reflux (heart burn), diarrhea, Crohns syndrome, and irritable bowel syndromes (IBS) and include disorders of the urinary tract, as for example “kidney stone pain” (urolithiasis), disorders of the gall fluid ducts, as for example “gall stone pains” (cholelithiasis and choledocholithiasis) and disorders of the smooth muscles of the airways as for example asthma, COPD and bronchitis. Numerous other smooth muscle disorders exist where anticholinergic drugs have therapeutic effects and such disorders are encompassed in the present invention.

The instant invention refers to the use of trospium in patients suffering mainly from urinary urge incontinence. Thos skilled in the art will realize that the present invention also refers to the treatment of patients suffering from Mixed Urinary Incontinence (MUI) since the patients suffering from MUI have symptoms of both urge urinary incontinence (UUI) and stress urinary incontinence (SUI). At present, no medication is able to effectively treat symptoms of both UUI and SUI.

Those skilled in the art of pharmacology, will realize that the compounds of the invention, being trospium and the active metabolites of trospium and possible salts and solvates thereof and possible prodrugs thereof, having certain pharmacological properties, such as antimuscarinic activity on various receptor types, calcium antagonistic activity, spasmolytic activity on various types of smooth muscle, etc., may be useful for other indications than those listed here.

What is claimed is:

1. A method for treating smooth muscle disorders in a human suffering from, or having a propensity for, a cardiac contractility disorder, comprising administering to said human a therapeutically effective amount of trospium or a pharmaceutically acceptable salt or solvate thereof or an active metabolite of trospium or a pharmaceutically acceptable prodrug, salt or solvate thereof.

2. A method for treating smooth muscle disorders in a human suffering from, or having a propensity for, a cardiac contractility disorder, comprising administering to said human a therapeutically effective amount of trospium or a pharmaceutically acceptable salt or solvate thereof or an active metabolite of trospium or a pharmaceutically acceptable prodrug, salt or solvate thereof, while avoiding drug-induced cardio-depressive side effects.

3. The method of claim 2, wherein said drug-induced cardio-depressive side effects are caused by medication used for treating said smooth muscle disorders.

4. The method of claim 1, wherein said smooth muscle disorder is a urinary voiding disorder.

5. The method of claim 4, wherein said urinary voiding disorder is urinary urge incontinence.

6. The method of claim 1 wherein said cardiac contractility disorder or the predisposition for said cardiac contractility disorder is caused by a disease that causes decreased cardiac contractility.

7. The method of claim 1 wherein said cardiac contractility disorder or the predisposition for said cardiac contractility disorder is caused by a drug that causes decreased cardiac contractility.

8. The method of claim 1, where said smooth muscle disorder is a disorder belonging to the group selected from urolithiasis, cholelithiasis and choledocholithiasis.

9. The method of claim 1, wherein the propensity for a cardiac contractility disorder is manifested as arterial hypertension.

10. The method of claim 1, wherein the propensity for a cardiac contractility disorder is manifested as arterial hypertension in an elderly patient.

11. The method of claim 1, wherein the propensity for a cardiac contractility disorder is manifested as obesity.

12. The method of claim 1, wherein the propensity for a cardiac contractility disorder is manifested as obesity and wherein said human is elderly.

13. The method of claim 1, wherein the propensity for a cardiac contractility disorder is manifested as pulmonary congestion or cardiac dyspnea.

14. The method of claim 1, wherein the propensity for a cardiac contractility disorder is manifested as pulmonary congestion or cardiac dyspnea and wherein said human is elderly.

15. The method of claim 1, wherein the propensity for a cardiac contractility disorder is manifested as a coronary heart disease.

16. The method of claim 1, wherein the propensity for a cardiac contractility disorder is manifested as a coronary heart disease and wherein said human is elderly.

17. A method of claim 1, wherein said individual has been suffering one or more cardiac infarcts.

18. The method of claim 1, wherein the propensity for a cardiac contractility disorder is manifested as alcohol abuse.

19. The method of claim 1, wherein the propensity for a cardiac contractility disorder is manifested as alcohol abuse and wherein said human is elderly.

20. The method of claim 1, wherein said active metabolite is a spiroalcohol metabolite.

21. The method of claim 1, wherein the amount of trospium, or a pharmaceutically acceptable salt or solvate thereof, or an active metabolite of trospium, or a pharmaceutically acceptable salt or solvate thereof, is administered from 1 mg to 240 mg per day.

22. The method of claim 1 wherein the amount of trospium, or a pharmaceutically acceptable salt or solvate thereof, or an active metabolite of trospium, or a pharmaceutically acceptable salt or solvate thereof, is administered from 10 mg to 60 mg per day.

23. The method of claim 1, wherein the amount of trospium or a pharmaceutically acceptable salt or solvate thereof, or an active metabolite of trospium or a pharmaceutically acceptable salt or solvate thereof thereof is administered together with a pharmaceutically acceptable carrier.

24. A method for treating smooth muscle disorders in a individual, comprising determining whether said individual suffers from or has a propensity for a cardiac contractility disorder, and if said determination is positive, administering to said individual a therapeutically effective amount of trospium or a pharmaceutically acceptable salt or solvate thereof or an active metabolite of trospium or a pharmaceutically acceptable prodrug, salt or solvate thereof.

25. The method of claim 24, wherein said smooth muscle disorder is a urinary voiding disorder.

26. The method of claim 25, wherein said urinary voiding disorder is urinary urge incontinence.
